(19) United States
(12) Patent Application Publication
(10) Pub. No.: US 2024/0221124 A1
(43) Pub. Date: Jul. 4, 2024

(54) SYSTEMS AND METHODS TO COMPOSITE PRE-ACQUIRED IMAGE DATA IN OCCLUDED AREAS OF A REAL-TIME IMAGE STREAM

(71) Applicant: Veran Medical Technologies, Inc., St. Louis, MO (US)

(72) Inventors: Christopher Lee, St. Louis, MO (US); Mark Hunter, St. Louis, MO (US)

(21) Appl. No.: 18/543,009

(22) Filed: Dec. 18, 2023

Related U.S. Application Data
(60) Provisional application No. 63/477,744, filed on Dec. 29, 2022.

(51) Int. Cl. G06T 5/50; G06V 10/25; G16H 30/20; G16H 30/40

(52) U.S. Cl. CPC G06T 5/50; G06V 10/25; G16H 30/20; G16H 30/40; G06T 2207/10132; G06T 2207/20221; G06V 2201/07

(57) ABSTRACT

Various aspects of methods, systems, and use cases may be used to generate and display a real-time a composite image, such as in a manner that extracts a non-occluded portion of a first image corresponding to an occluded portion of a second image and replaces the occluded portion with the non-occluded portion. In some examples, a graphical representation of an instrument may be generated on the composite image.

### PRIORITY CLAIM

This application claims the benefit of priority to U.S. Provisional Patent Application Ser. No. 63/477,744, filed Dec. 29, 2022, the contents of which are incorporated herein by reference.

### BACKGROUND

Prior to, or during, medical procedures, imaging may be used to view portions of the patient's body. For example, imaging may be used to view internal organs, tissues, and other anatomy of the patient. Imaging techniques and devices may include, for example, ultrasound imaging or computerized tomography (CT) imaging. Ultrasound imaging uses high-frequency sound waves to produces images of the inside the patient's body. CT imaging combines a series of x-ray images taken from a plurality of angles around the body to generate cross-sectional images of the inside of the patient's body.

Such imaging techniques may be used for patients with solitary pulmonary nodules (SPNs), which may exist just outside of an airway wall, and which may be identified on CT images. In many cases, upon identifying a SPN in a CT image set, a pulmonologist will perform a biopsy to obtain a tissue sample for pathology. This is because although most SPNs are benign, some represent early stage lung cancer which left untreated could be fatal.

A patient with SPNs may undergo an endobronchial ultrasound (EBUS) procedure to obtain a biopsy sample. An EBUS procedure may be used to diagnose various lung problems, including inflection, disease, or cancer. During the EBUS procedure, a needle may be used to obtain tissue or fluid samples while the clinician views a real-time ultrasound image to confirm the needle is entering and sampling the SPN. Real-time imaging via an EBUS device is often needed due to the dynamic nature of lungs throughout a respiratory cycle and the small size of the SPNs. For example, an SPN may be less than 2 cm in diameter and may move more than 2 cm throughout the respiratory cycle. Accordingly, although a pre-operative CT scan may reveal the presence of SPNs, such static scans do not provide confirmation as to the present real-time location of SPNs.

### SUMMARY

The invention discloses systems and methods for compositing pre-acquired image data into occluded areas of a real-time image stream. In one aspect, the system identifies an occluded portion within real-time ultrasound data and fills or replaces the occluded portion with image data from a source other than the real-time ultrasound data.

For example, previously acquired ultrasound images captured just prior to an occlusion event may be used to enhance the real-time stream. The system can analyze one or more ultrasound images captured seconds earlier when the now-occluded portion was not obstructed. It then composites the matching non-occluded ultrasound data into the occluded area. This enables clinicians to view a full representation of tissue as if no occlusion occurred.

Occlusions can be caused by instruments like biopsy needles or ablation devices entering the field of view. They may also be caused by air bubbles that interrupt ultrasound transmission. The system detects occluded portions by analyzing pixel data and identifying areas lacking tissue characteristic information. It may also use known instrument signatures or air bubble signatures to identify occluded areas.

In another embodiment, the system composites pre-acquired CT or other modality data into occluded areas after processing to conform it to the real-time ultrasound data. For example, CT data representing an occluded area can be transformed to appear like ultrasound data before compositing into the live stream.

The system can additionally track position and orientation of images using techniques like electromagnetic tracking or respiration gating. This navigation data helps align non-occluded portions of historical images with live occluded areas. It also allows for warping and deforming composited data to match real-time anatomy.

The systems and methods disclosed enable clinicians to view a full, rich image with valuable tissue information during procedures despite real-time occlusions that normally obstruct portions of the image.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is a schematic diagram that illustrates an example internal medical imaging system for use within a lumen of a patient.

FIG. 1B is a schematic diagram that illustrates an example internal medical imaging system for use within a lumen of a patient with an instrument extended from the imaging system.

FIG. 2A illustrates an example of a method of generating a composite image.

FIG. 2B illustrates an example set of images being processed into a composite image using the method of FIG. 2A.

FIG. 3A illustrates an example of a method of generating a composite image using multiple imaging modalities.

FIG. 3B illustrates an example set of images being processed into a composite image using the method of FIG. 3A.

FIG. 4A illustrates an example of a method of generating a composite image with a graphical indication.

FIG. 4B illustrates an example set of images being processed into a composite image using the method of FIG. 4A.

FIG. 5A a schematic diagram that illustrates a nonoccluded field of view from an internal medical imaging system.

FIG. 5B a schematic diagram that illustrates a needle causing an occluded portion of the field of view of the internal medical imaging system of FIG. 5A.

FIG. 5C a schematic diagram that illustrates the field of view of FIG. 5A, with a corresponding occluded portion identified by a computing device.

FIG. 5D a schematic diagram that illustrates a composite image created from the field of view of FIG. 5A and FIG. 5B.

FIG. 6 illustrates a block diagram of an example machine upon which any one or more of the techniques discussed herein may perform.

### DETAILED DESCRIPTION

Ultrasound (US) imaging is used in the respiratory area to positively identify a target area from which to acquire a biopsy sample because the ultrasound imaging can show both tissue and an instrument (e.g., biopsy needle or sheath) in real time. However, the instrument often interrupts the transmission of ultrasound energy to tissue beyond the instrument in relation to the source of the ultrasound energy (e.g., US transducer). Thus, when the instrument is present within the field of view of an ultrasound image, an occluded area beyond the instrument in relation to the transducer may lack information indicating tissue characteristics while a physician directs the instrument toward the target area in order to biopsy. Accordingly, the inventors have recognized that an imaging system that can continue to show a rich image of tissue within such an occluded area is desirable.

In addition to instruments obstructing ultrasound transmission and impacting image quality, the inventors have recognized that certain disease states can occlude ultrasound transmission and impact imaging during a procedure. For example, patients with COPD have obstructed airflow from the lungs. The obstruction of the lungs can cause air bubbles to be caught in the tissue. When imaging lungs of patients with COPD, air bubbles reduce the quality of the images. When imaging with ultrasound devices, the sound waves hit the air bubble(s) and prevent imaging of the tissue beyond the air bubble(s).

To mitigate these drawbacks, the inventors have developed a system that can identify an occluded portion within real-time ultrasound data and fill the occluded portion, or replace the occluded portion, with image data from some source other than the real-time ultrasound data. For example, previously acquired image data may be used. The previously acquired image data may be used to enhance portions of an image that is being acquired in real time and that has become occluded or includes an occluded portion. Such an occlusion can be caused by an air bubble, an instrument (e.g., a biopsy needle or ablation device) entering the field of view of the imaging device (e.g., an ultrasound transducer), or a combination thereof.

More specifically, the concept discussed herein is a system that is designed to utilize previously acquired image data to enhance portions of an image that is being acquired in real time and that has become occluded (e.g., due to an instrument entering the field of view of an imaging device such as an ultrasound transducer). An exemplary scenario in which the proposed concept would be beneficial is an ultrasound assisted biopsy. During such a procedure, a doctor may navigate an endobronchial ultrasound (EBUS) tissue sampling device toward a target nodule (e.g., a preidentified tissue from a CT scan for which a biopsy sample is desired). Upon positively identifying the target nodule in the ultrasound image, the doctor may advance a sheath enshrouding a sampling needle from a working channel of the EBUS tissue sampling device. The sheath may be a flexible plastic sleeve which prevents the sampling needle from damaging the EBUS tissue sampling device at side exit having a ramp (e.g., that deflects the sampling needle away from a longitudinal axis of the sampling device toward the target nodule beyond an airway wall).

It can be appreciated that upon the instrument (e.g., sampling needle and/or sheath) being extended from the side exit it will enter the field of view of the ultrasound transducer. Although this is desirable and by design to enable the doctor to visually see the instrument within the generated ultrasound images in real-time, one drawback is that the instrument may occlude an area of the image that lies behind the instrument relative to the transducer. The proposed concept is intended to mitigate this drawback by identifying this occluded portion and filling it in with image data from some source other than the real-time ultrasound data.

In some embodiments, the system may identify the occluded portion and then utilize previously acquired ultrasound images that were obtained just prior to the instrument causing the occluded portion. For example, the system may analyze one or more ultrasound images that were captured a few seconds earlier when the occluded portion was not yet occluded, and composite a portion of these ultrasound images that matches the occluded portion onto the occluded portion in substantially real-time. In this way, despite the real-time ultrasound images having the occluded area, the doctor is still shown a graphical representation of tissue within the entire field of view of the ultrasound as if the instrument did not have the effect of occluding the image.

In some embodiments, the system may composite image information from another imaging modality into the occluded portion. For example, previously acquired CT data that represents the occluded portion may be processed and caused to appear like ultrasound data and then composited into the occluded portion of the real-time ultrasound image.

The above discussion is intended to provide an overview of subject matter of the present patent application. It is not intended to provide an exclusive or exhaustive explanation of the invention. The description below is included to provide further information about the present patent application.

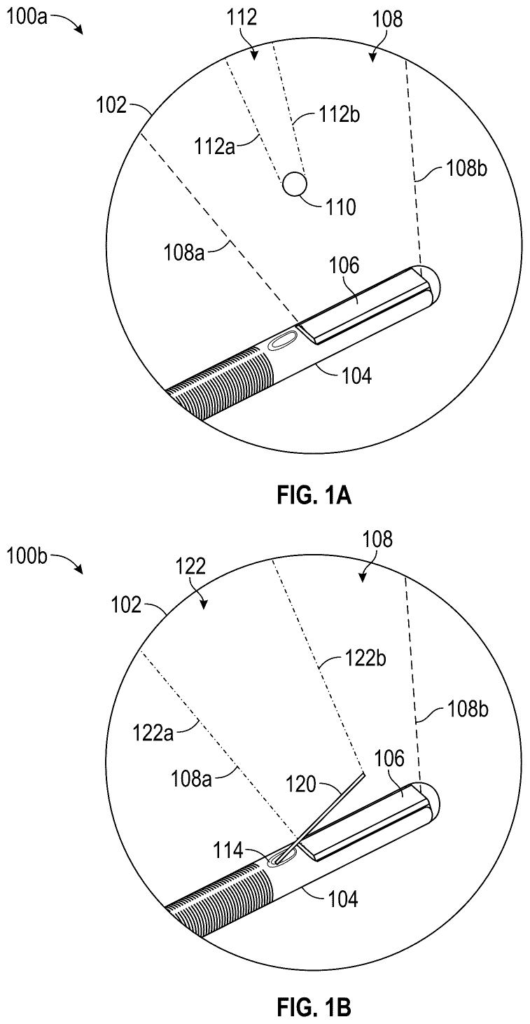

FIG. 1A illustrates a schematic diagram 100a of an example internal medical imaging system 104 with an imaging sensor 106. In an example, the internal medical imaging system 104 is inserted into a target area 102 of a patient.

In an example, the internal medical imaging system 104 is an endobronchial ultrasound (EBUS) tissue sampling device. In an example, the internal medical imaging system 104 is used to navigate towards and image a target nodule prior to tissue sampling. In an example, the target nodule is a preidentified tissue for which a biopsy sample is desired. In an example, ultrasound (US) imaging is used in the respiratory area to positively identify a target area from which to acquire a biopsy sample because the ultrasound imaging shows both tissue and an instrument (e.g., needle or sheath) in real time.

In an example, the imaging sensor 106 is positioned at a distal end of the internal medical imaging system 104. In an example, the imaging sensor 106 extends along a portion of the distal end. In an example, the imaging sensor 106 is substantially flat. In an example, the imaging sensor 106 is an ultrasound transducer. In an example, the imaging sensor 106 provides a field of view 108 to an imaging display.

During imaging, an occlusion 110 in the field of view 108 causes an occluded portion 112 in the field of view 108. The field of view 108 extends from a proximal boundary 108a to a distal boundary 108b. In an example, the occlusion 110 is an air bubble causing the occluded portion 112. As illustrated in FIG. 1A (and FIG. 1B for that matter), the occluded portion extends from a proximal boundary 112a to a distal boundary 112b. The occluded portion 112 is caused by an interruption of the transmission of ultrasound energy to tissue beyond the occlusion 110 in relation to the imaging sensor 106 (e.g., US transducer). For example, ultrasonic waves transmitted from the imaging sensor 106 (e.g., an ultrasound transducer) may propagate through tissues until reaching a boundary between tissue and the air bubble (which in this example is the occlusion 110) at which a substantial amount of the ultrasound energy is reflected back toward the imaging sensor 106 with an insubstantial amount continues propagating away from the imaging sensor 106 and into the air bubble. The occluded portion 112 can lack information indicating tissue characteristics. Such a lack of information can affect the ability of a physician to direct the internal medical imaging system 104 toward a target area of the patient. Therefore, as described in further detail below, a display device, or computing device thereof, connected to the internal medical imaging system 104 uses previously acquired imaging data from the field of view to create a composite image including the previously acquired image and an image collected in real-time. The computing devices uses a portion of the previously acquired image that aligns with the occluded portion 112 to replace the occluded portion, such that the clinician is provided with a full image of the field of view, even when a portion is occluded.

FIG. 1B illustrates a schematic diagram 100b of an example internal medical imaging system 104 with an instrument 120 extended from the internal medical imaging system 104 in the target area 102 of a patient.

In an example, the instrument 120 is advanced through the internal medical imaging system 104 via a working channel. In an example, the instrument 120 is extended from an exit port 114 of the internal medical imaging system 104. In an example, the exit port 114 includes a ramp, such that the instrument 120 extends from the internal medical imaging system 104 at an angle. In an example, the instrument 120 is a sheath. In an example, the sheath is a flexible plastic sleeve. In another example, the instrument 120 is a needle. In an example, the needle is a sampling needle for capturing biopsies of target tissues (e.g., which may be preidentified via a pre-operative image source such as a CT scan).

As another example, the instrument 120 includes both a sheath and a needle. As an example, the sheath enshrouds the needle. As another example, the sheath is partially extended out of the internal medical imaging system 104 prior to the needle being extended from the exit port, such that the sheath protects the exit port 114.

During imaging, the extension of the instrument 120 out of the internal medical imaging system 104 causes an occluded portion 122 in the field of view 108. The occluded portion 122 is caused by an interruption of the transmission of ultrasound energy to tissue beyond the instrument 120 in relation to the imaging sensor 106 (e.g., US transducer). While it can be advantageous for the user to see the instrument 120 within the field of view 108, the occluded portion 122 caused by the instrument 120 may lack information indicating tissue characteristics beyond the instrument 120. In this example, the field of view 108 and the occluded portion 122 share a common proximal boundary-proximal boundary 108a and proximal boundary 122a—while the distal boundary 122b of the occluded portion 122 will track distally as the instrument 120 is extended into the field of view 108. Such a lack of information can affect the ability of a physician to direct the internal medical imaging system 104 toward a target area of the patient and/or may affect the ability of the physician to direct the instrument 120 to a target object within the target area.

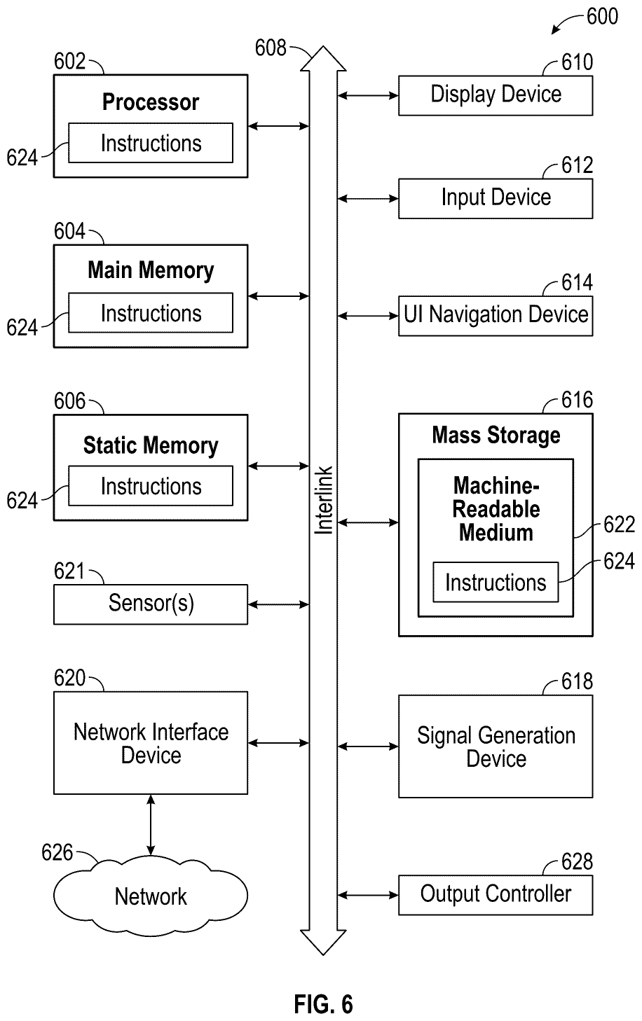

In an example, the internal medical imaging system 104 of FIGS. 1A and 1B includes or is connected to a display device to display an image of the field of view 108 obtained by the imaging sensor 106. In an example, the internal medical imaging system 104 is connected to a computing device. The computing device is capable of image processing (e.g., the machine of FIG. 6).

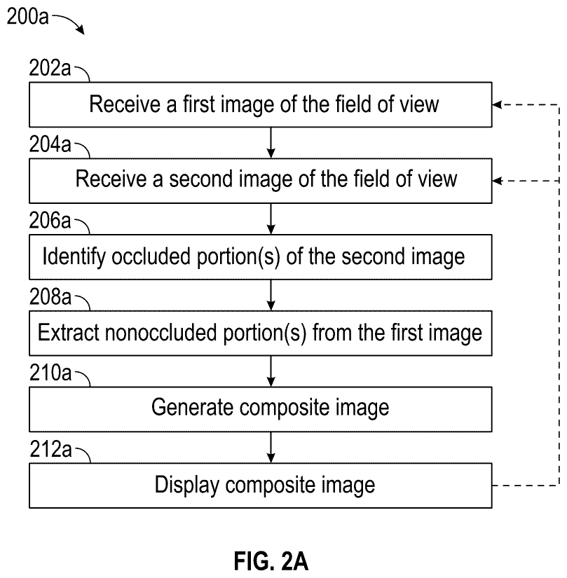

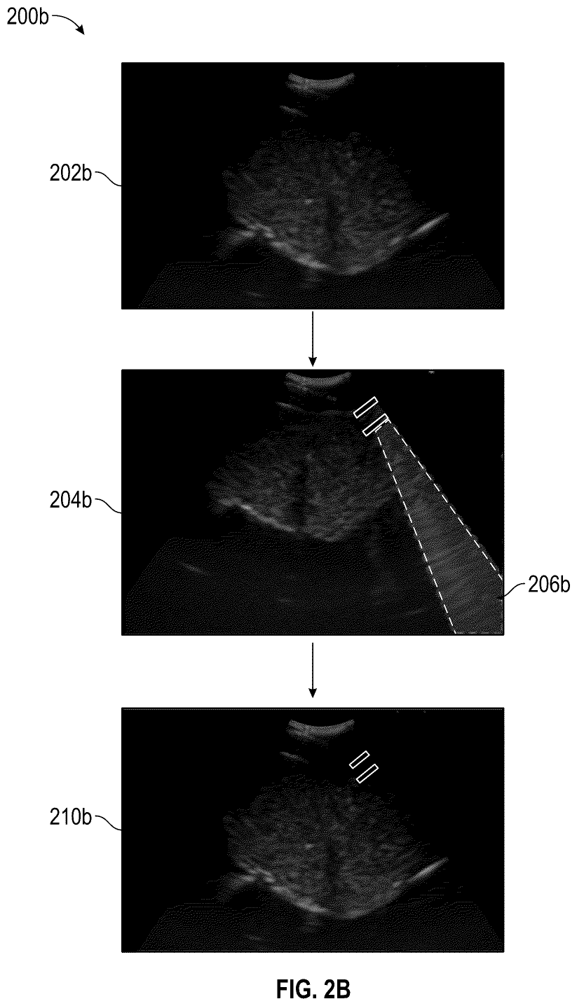

FIG. 2A illustrates an example of a method 200a of generating a composite image. FIG. 2B illustrates an example set of images 200b being processed into a composite image using the method of FIG. 2A. In an example, the method 200a can be performed by a computer device (e.g., the machine of FIG. 6) communicatively coupled to the EBUS. The computer device includes processing circuitry for conducting various image processing tasks discussed in conjunction with method 200a. Further, the computer device can include an output display, such as a monitor to display the ultrasound image and various additional user interface elements, among other things.

At the beginning of method 200a, such as at the beginning of a procedure, the imaging device (e.g., the internal medical imaging system 104) is positioned to the desired field of view. In an example, once the imaging device is in position, the imaging device is rotated to collect a plurality of images in a plurality of tracked positions. In some embodiments, the imaging device 104 is an EBUS device with navigation capabilities such as, for example, embedded sensor coils that when placed within an electromagnetic field generated during the procedure provide signals to a navigation system that allows for precise tracking of position and orientation within the patient. The navigation system of the imaging device can utilize electromagnetic (EM) tracking techniques, that allow for tracking the imaging device within six degrees of freedom. In an example, the device is rotated between 15 degrees and 360 degrees. In an example, images are created between the images collected at different degrees using interpolation. In an example, the tracked positions include a position of the imaging device and an orientation of the imaging device.

In an example, the imaging system, or another connected system, monitors and collects respiratory information associated with the image. For example, the portion of the respiratory cycle is captured. In an example, the respiratory information is received via a respiratory gate tracking device. A respiratory gate tracking device can indicate where within a respiratory cycle the patient is so that the system can adjust the shape and location of a 3D model of the patient's lung to account for breathing. An example respiratory gate tracking device is discussed within U.S. Pat. No. 10,617,324, titled “Apparatuses and methods for endobronchial navigation to and confirmation of the location of a target tissue and percutaneous interception of the target tissue,” which is hereby incorporated by reference in its entirety. The respiratory gate tracking device can include a plurality of markers that can move and change orientation and shape during movement of the patient caused by different stages of the respiratory cycle. In an example, the computing device uses the distance between a set of the plurality of markers at a given time to determine the stage of the respiratory cycle. In an example, the markers are visible in images. For example, the computing device uses the images of the markers to determine the distance between the markers to determine the current stage of the respiratory cycle. Accordingly, in an example, within certain parts of the anatomy, the respiratory cycle is factored into the navigation data to further enhance the orientation and position information used for imaging.

At 202a, the computing device receives a first image 202b of the field of view. The computing device receives the first image 202b captured from the imaging sensor 106 of the internal medical imaging system 104. In an example, the first image 202b is of the target area of the patient. For example, the first image 202b may include within a field of view a target tissue for which a biopsy sample and/or ablation treatment is desired such as a solitary pulmonary nodule (SPN) located just outside of and adjacent to an airway. The first image 202b may be free of any occlusions. In an example, the first image 202b includes a tracked position. In an example, a plurality of first images 202b are received. The EBUS device is a real-time imaging device, that provides a stream of images. Accordingly, in these examples, discussion of “a first image” is used generally to reference a first imaging state (e.g., a first imaging state that does not include an occlusion). Thus, when the method 200a is discussed as “receiving a first image”, this can be interpretated as a shorthand for a stream of images in a first imaging state. In certain scenarios, the computing device may operate on a single image or a stream of images with a real-time or near real-time update to the display device. For convenience, much of the method is described in terms of individual images, but the operations are performed in real-time on a stream of images.

At 204a, the computing device receives a second image 204b of the field of view. The second image 204b is received after the first image 202b. The second image 204b includes an occluded area 206b. In an example, the second image 204b is received in real time. In an example, the computing device receives a plurality of second images 204b. In an example, the second image 204b is received immediately following the first image 202b. In an example, the second image 204b includes the tracked position. The second image 204b which includes the occluded area 206b may be a sequential image from the same image stream in which the first image 202b is received. For example, each of the first image 202b and second image 204b may be provided from an EBUS sampling device to an ultrasound image processor within the same image stream during a single procedure.

At 206a, the computing device identifies one or more occluded portions 206b of the second image 204b. In an example, the computing device identifies the one or more occluded portions 206b by assessing pixels of the second image 204b. In an example, the computing device determines that the pixels in the occluded portion 206b are all black or otherwise contain data that is clearly not from imaged tissue. In another example, the computing device compares a set of pixel data from the second image 204b to a baseline value of pixel data. The baseline value of pixel can be set to indicate that the pixel is occluded. In an example, the computing device determines that a portion of the second image 204b is occluded when a subset of the set of pixel data is below the baseline value. In an example, instead of assessing individual pixels, the computing device compares pixel data in groups of pixels. The computing device can determine that beyond a certain area is the occluded portion 206b. For example, the computing device determines that beyond a certain line of data, additional information is not being collected. In an example, the computing device detects an immediate change in quality past an occlusion causing the occluded portion 206b (e.g., collected ultrasound data may indicate that beyond a certain depth at which a large ultrasound reflection occurs very little ultrasound data is being reflected back to the transducer). The occluded image portion may also be identified through identification of an instrument within the image. For example, a biopsy needle will typically produce a line of bright white pixels within an ultrasound image, as the metal needle reflects ultrasound efficiently. In an example, the computing device examines the second image 204b in segments based on imaging rays and identify the occluded portion 206b when data is expected in the second image 204b, but no data is collected (e.g., the second image 204b is producing zeros beyond a certain point). In an example, the computing device detects the occluded portion 206b by detecting an air bubble in the field of view of the second image 204b. The computing device detects the air bubble using the techniques described above. In an example, the computing device detects the air bubble by applying an image processing algorithm trained to detect an imaging signature of an air bubble. Image processing algorithms can be utilized to identify these known artifacts and can then interpolate the occlusion area. In certain examples, imagine processing algorithms can look for changes in contrast, such as areas with little to no contrast changes are likely to be occluded areas of an image.

At 208a, the computing device extracts the nonoccluded portion of the field of view from the first image 202b. The nonoccluded portion from the first image 202b substantially corresponds with the occluded portion 206b of the second image 204b. In some examples, each of the first image 202b and the second image 204b includes position and orientation data (e.g., tracked position) associated with the image. In an example, the computing device can utilize information about the field of view and the tracked position to understand a relationship between the first image 202b and the second image 204b. As another example, the computing device can utilize a plurality of first images to determine the nonoccluded portion of the first of view to extract from a first image 202b that has a substantially similar tracked position as the second image 204b. In certain examples, the nonoccluded portion may be identified within a first image 202b that was not taken at precisely the same tracked position as the second image 204b. In such examples, position and orientation data associated with the second image 204b may be used to identify precisely where in 3D space the patient's tissue is occluded and position and orientation data associated with the first image 202b may be used to identify non-occluded image data corresponding to this 3D space for use in compositing into the second image 204b.

At 210a, the computing device generates a composite image 210b. In an example, the computing device generates a composite image by replacing the occluded portion 206b of the second image 204b with the nonoccluded portion of the first image 202b. For example, the occluded portion 206b can be extracted from the second image 204b and replaced by the nonoccluded portion of the first image 202b. In another example, the computing device generates a composite image by overlaying the nonoccluded portion of the first image 202b on the occluded portion 206b of the second image 204b. The composite image may be generated in substantially real-time. The system may determine a scale and pose adjustment factor between the real time ultrasound stream (e.g., the second image 204b) and the previously acquired ultrasound image (e.g., the first image 202b) and transform a portion of the previously acquired ultrasound image (e.g., the first image 202b) based on the scale and pose adjustment. The nonoccluded portion of the first image 202b may be deformed to align with the occluded portion 206b of the second image 204b. In certain examples, the nonoccluded portion may be identified within a first image 202b that was not taken at precisely the same navigational position and orientation as the second image 204b. In these examples, the computing device can either transpose the nonoccluded portion of the first image based on a comparison of navigation data between the first image 202b and the second image 204b, or interpolate between the nonoccluded portion of the first image 202b and the occluded portion 206b of the second image 204b based on the navigation data for each image. In the case of transposing the data, the nonoccluded portion is from a first image 202b that is sufficiently close in position and orientation, that the nonoccluded portion just needs to be shift into the position of the occluded portion 206b of the second image 204b. Interpolation may need to occur when the position and orientation between the first image 202b and the second image 204b is more significant (but still within the range that allows for representative adjustment of the nonoccluded portion).

At 212a, the computing device displays the composite image 210b. Despite the real-time second image having the occluded portion 206b, the user is now shown an image representation of tissue within the entire field of view as if the occluded portion 206b did not have the effect of occluding the second image 204b. If the computing device does not receive an image with an occluded portion 206b, the first image 202b is displayed unaltered by the processing described here. In an example, the computing device enables the user to switch between multiple image display modes. For example, the computing device may include a first mode that generates a composite image when an occluded portion is detected and a second mode that displays an unaltered real-time image. In some embodiments, the system may be configured to display graphical user interface elements (GUIs) indicating a delineation between a portion of the image stream which corresponds to unaltered real-time image data and which portion corresponds to composited image data that supplements the occluded portion. Exemplary such GUIs include, but are not limited to, a colored boundary line delineating the composited image portion, or, displaying the composited image portion is a different color or shade as compared to the unaltered image portion, or both.

Method 200a operates on the computing device in real-time and continuously throughout the procedure. For example, while the instrument is extended into the field-of-view second images 204b continue to be collected with an occluded portion 206b, and method 200a operates in real-time to generate and display composite images 210b using a portion of the first image 202b extracted to replace the occluded portion 206b of the second image 204b. As another example, as the imaging device moves through a passageway, as an air bubble sits in the passageway, second images 204b continue to be collected with an occluded portion 206b, and method 200a operates in real-time to generate display composite images 210b using a portion of the first image 202b extracted to replace the occluded portion 206b of the second image 204b. As another example, first images 202b will continue to be collected until a second image 204b with an occluded portion 206b is identified. First images 202b and second images 204b may be continuously received and new composite images may be displayed throughout the procedure. As method 200a operates, the first image 202b may be selected such that it is an image received just prior to the second image 204b, which includes the occluded portion 206b. Among other benefits, the techniques described herein enable a user to see the entire boundary of a target tissue (e.g., a SPN) even while a sampling needle or ablation device is inserted partially into the target tissue to obtain a biopsy or to perform an ablation procedure. In this way, the user is provided with valuable information within the displayed images which conventional medical imaging systems (e.g., EBUS systems) lack capabilities to provide.

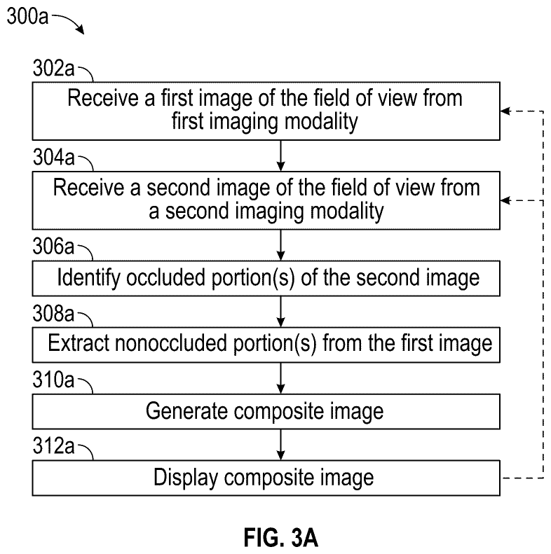

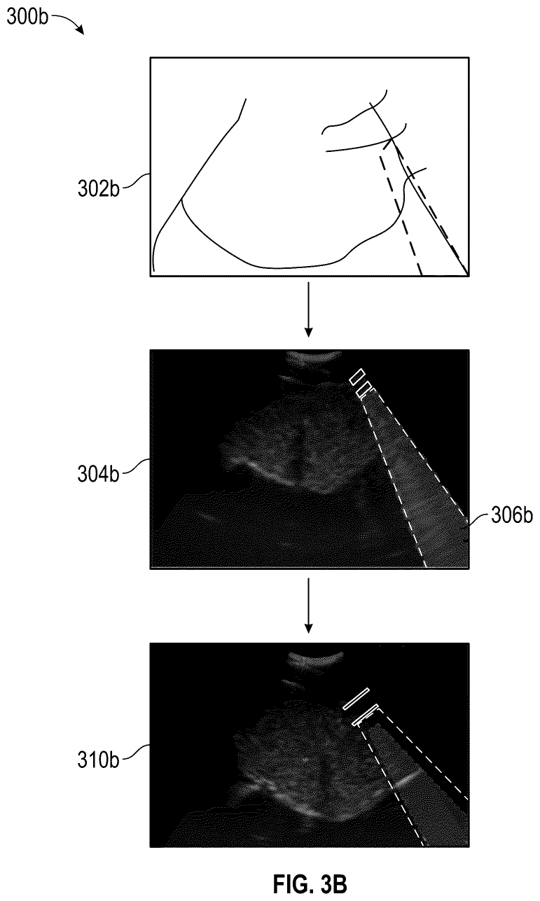

FIG. 3A illustrates an example of a method 300a of generating a composite image using multiple imaging modalities. FIG. 3B illustrates an example set of images 300b being processed into a composite image using the method of FIG. 3A. In an example, the method 300a can be performed by a computer device (e.g., the machine of FIG. 6) communicatively coupled to the EBUS. The computer device includes processing circuitry for conducting various image processing tasks discussed in conjunction with method 300a. Further, the computer device can include an output display, such as a monitor to display the ultrasound image and various additional user interface elements, among other things.

When collecting, capturing, or receiving images or image data, the images may be registered. For example, the images may be registered in image space in relation to the patient space. The registration can include orientation, position, location, and/or rotation of the image in relation to the patient. For example, various points in the images or the patient may be identified to register the image. In an example, the points are easily identifiable anatomical landmarks. The points can be used to establish point based registration between images, such that the image data includes information pertaining to the orientation, position, location, and/or rotation of the image in relation to the patient or other images. If the image sensor is moved, the movement of the points may be used to determine updated information regarding orientation, position, location, and/or rotation of the image in relation to the patient or other images. In an example, when collecting, capturing, or receiving images or image data, the images can be registered using four-dimensional (4D) data. During 4D registration, the image data is collected to render a three-dimensional (3D) image (e.g., a volumetric image) of the target area. The 3D image further includes motion resulting from movement of a patient's anatomy such as that caused by the patient's respiratory cycle and/or the patient's heartbeat, thus producing 4D data and a 4D image.

At 302a, the computing device receives a first image 302b of the field of view from a first imaging modality. In an example, the computing device receives the first image 302b from a computerized tomography (CT) device. In an example, the first image 302b is of the target area of the patient. For example, the first image 302b may include within a field of view a target tissue for which a biopsy sample and/or ablation treatment is desired such as a solitary pulmonary nodule (SPN) located just outside of and adjacent to an airway. The first image 302b may be free of any occlusions. In an example, a plurality of first image 302b may be obtained in a plurality of tracked positions. For example, the first images 302b is CT scans collected in 1 mm slices. In an example, the computing device interpolates between the slices to create a new first image 302b. In an example, the first image 302b includes anatomical landmarks and/or indicates a current anatomical state. For example, the anatomical state includes a respiratory state. The first image 302b can be collected during total lung capacity and/or tidal volume expiration. In an example, the computing device receives a plurality of first images 302b.

Prior to receiving a second image 304b, such as at the beginning of a procedure, the imaging device (e.g., the internal medical imaging system 104) is positioned in the desired field of view. In an example, once the imaging device is in position, the imaging device may be rotated to collect a plurality of images in a plurality of tracked positions. In an example, the imaging device 104 is an EBUS device with navigation capabilities such as, for example, embedded sensor coils that when placed within an electromagnetic field generated during the procedure provide signals to a navigation system that allows for precise tracking of position and orientation within the patient. The navigation system of the imaging device can utilize electromagnetic (EM) tracking techniques, that allow for tracking the imaging device within six degrees of freedom. In an example, the imaging device is rotated about the longitudinal axis of the device. The imaging device may be rotated between 15 degrees and 360 degrees. The longitudinal rotation of the imaging device can generate a series of fanned out images. In an example, the computing device interpolates between the images collected at different degrees to create additional images. In an example, the tracked positions include a position of the imaging device and an orientation of the imaging device.

In an example, the imaging system, or another connected system, monitors and collects respiratory information associated with the image. For example, the portion of the respiratory cycle is captured. In an example, the respiratory information is received via a respiratory gate tracking device. For example, the respiratory gate tracking device includes a plurality of markers that can move and change orientation and shape during movement of the patient caused by different stages of the respiratory cycle. In an example, the computing device uses the distance between a set of the plurality of markers at a given time to determine the stage of the respiratory cycle. In an example, the markers are visible in images. For example, the computing device uses the images of the markers to determine the distance between the markers to determine the current stage of the respiratory cycle. Accordingly, in an example, within certain parts of the anatomy, the respiratory cycle is factored into the navigation data to further enhance the orientation and position information used for imaging.

At 304a, the computing device receives a second image 304b of the field of view from a second imaging modality. The second image 304b is received after the first image 302b. The second image 302b can be of the target area of the patient. The second image 304b includes an occluded area 306b. In an example, the second image 304b is received in real time. The second image modality is the imaging sensor 106 of the internal medical imaging system 104. In an example, the computing device receives a plurality of second images 304b. The second image 304b is received following the first image 302b. For example, the first images 302b may collected prior to the procedure in which the second image 304b is received. The EBUS device is a real-time imaging device, that provides a stream of images. Accordingly, in these examples, discussion of “a second image” is used generally to reference a second imaging state (e.g., a second imaging state that includes an occlusion). Thus, when the method 300a is discussed as “receiving a second image”, this can be interpretated as a shorthand for a stream of images in a second imaging state. In certain scenarios, the computing device may operate on a single image or a stream of images with a real-time or near real-time update to the display device. For convenience, much of the method is described in terms of individual images, but the operations are performed in real-time on a stream of images. In an example, the second image 304b includes tracked position. In an example, the second image 304b includes anatomical landmarks and/or indicates a current anatomical state. For example, the anatomical state can include a respiratory state.

At 306a, the computing device identifies one or more occluded portions 306b of the second image 304b. In an example, the computing device identifies the one or more occluded portions 306b by assessing pixels of the second image 304b. In an example, the computing device determines that the pixels in the occluded portion 306b are all black or otherwise contain data that is clearly not from imaged tissue. In another example, the computing device compares a set of pixel data from the second image 304b to a baseline value of pixel data. The baseline value of pixel can be set to indicate that the pixel is occluded. In an example, the computing device determines that a portion of the second image 304b is occluded when a subset of the set of pixel data is below the baseline value. In an example, instead of assessing individual pixels, the computing device compares pixel data in groups of pixels. The computing device may determine that beyond a certain area is the occluded portion 306b. For example, the computing device can determine that beyond a certain line of data, additional information is not being collected. In an example, the computing device detects an immediate change in quality past an occlusion causing the occluded portion 306b (e.g., collected ultrasound data may indicate that beyond a certain depth at which a large ultrasound reflection occurs very little ultrasound data is being reflected back to the transducer). The occluded image portion may also be identified through identification of an instrument within the image. For example, a biopsy needle will typically produce a line of bright white pixels within an ultrasound image, as the metal needle reflects ultrasound efficiently. In an example, the computing device examines the second image 304b in segments based on imaging rays and identify the occluded portion 306b when data is expected in the second image 304b, but no data is collected (e.g., the second image 304b is producing zeros beyond a certain point). In an example, the computing device detects the occluded portion 306b by detecting an air bubble in the field of view of the second image 304b. The computing device can detect the air bubble using the techniques described above. The computing device may detect the air bubble by applying an image processing algorithm trained to detect an imaging signature of an air bubble. Image processing algorithms can be utilized to identify these known artifacts and can then interpolate the occlusion area. In certain examples, imagine processing algorithms can look for changes in contrast, such as areas with little to no contrast changes are likely to be occluded areas of an image.

At 308a, the computing device extracts a nonoccluded portion of the field of view from the first image 302b. The nonoccluded portion from the first image 302b substantially corresponds with the occluded portion 306b of the second image 304b. The nonoccluded portion of the first image 302b is acquired prior to the second image 304b. Each of the first image 302b and the second image 304b includes position and orientation data (e.g., tracked position) associated with the image. In an example, the tracked position includes respiratory state information. In an example, the computing device utilizes information about the field of view and the tracked position to understand a relationship between the first image 302b and the second image 304b. As another example, the computing device utilizes a plurality of first images to determine the nonoccluded portion of the first of view to extract from a first image 302b that has a substantially similar tracked position as the second image 304b. In certain examples, the nonoccluded portion may be identified within a first image 302b that was not taken at precisely the same tracked position as the second image 304b. In such examples, position and orientation data associated with the second image 304b may be used to identify precisely where in 3D space the patient's tissue is occluded and position and orientation data associated with the first image 302b may be used to identify non-occluded image data corresponding to this 3D space for use in compositing into the second image 304b.

Prior to extracting the nonoccluded portion from the first image 302b, the first image 302b is deformed to correspond with the second image 304b. In this example, because the first image 302b is obtained from a different imaging modality as compared to the second (real-time) image 304b, the first image 302b must be adjusted (deformed) to appropriately correspond with the second image 304b. Alternatively or additionally, the nonoccluded portion may be deformed after the nonoccluded portion is extracted and while a composite image 310b is being generated at 310a. In an example, the first image 302b is deformed to conform with the current anatomical state of the second image 304b. Such deformation can include identifying anatomical landmarks in the first image 302b and the second image 304b. After identifying the anatomical landmarks, the first image 302b may be deformed such that the anatomical landmarks in the first image 302b align with the anatomical landmarks of the second image 304b. In an example, the anatomical landmarks include vessels.

In an example, the computing device proceeds with method 300a when a second image 304b has been collected with a designated respiratory state. In an example, the designated respiratory state is total lung capacity and/or tidal volume expiration. As another example, the first image 302b can be collected at the designated respiratory states (e.g., total lung capacity and tidal volume expiration) and deformed using a deformation matrix to interpolate data and imaging between the designated respiratory states (e.g., total lung capacity and tidal volume expiration). For example, the deformation matrix can be used when the first image 302b and the second image 304b are captured during different respiratory states. Additionally or alternatively, both the first image 302b and the second image 304b may be collected in a predetermined respiratory state (e.g., total lung capacity and/or tidal volume expiration), such that all deformation occurs with images collected in the same respiratory state. For example, the image capture can be triggered based on the respiratory state. As another example, images are continually collected, but only images that are collected at the designated respiratory state are used to generate composite images. As another example, the first image 302b may be selected from a plurality of first images such that the selected first image 302b was captured during the same respiratory state as the second image 304b.

In an example, using a plurality of two-dimensional (2D) images obtained by the image sensor, a three-dimensional (3D) model of the target area is generated. Using the 3D model, newly captured or received images can be matched to the 3D model to determine the location or position of the image. Using the location or position information, portions (e.g., nonoccluded portions) of the first image 302b can be extracted to align with the occluded portion 306b of the second image 304b. The extracted portion can overlay the occluded portion 306b, can be enhanced, can replace the occluded portion 306b, or a combination thereof. As another example, the computing device simulates image data to replace the occluded portion 306b with information that would be provided had the portion not been occluded. As another example, the computing device digitally reconstructs CT image data to virtually fill or replace the occluded portion 306b of the second image 304b when creating the composite image 310b. In an example, when collecting, capturing, or receiving images or image data, the images are registered using four-dimensional (4D) data. For example, the image data is collected to render a three-dimensional (3D) image (e.g., a volumetric image) of the target area. The 3D image can further include motion resulting from the respiratory cycle, thus producing 4D data and a 4D image.

In an example, the CT image is converted to an ultrasound image. In some cases, converting the CT image to the ultrasound image may create a crisper image or provide a more fluid composite image as both images are ultrasound images. For example, previously acquired CT data that represents the occluded portion (e.g., a nonoccluded portion of the CT image data) can be processed and caused to appear like ultrasound data and then composited into the occluded portion of the real-time ultrasound image.

At 310a, the computing device generates a composite image 310b. In an example, the computing device generates a composite image by replacing the occluded portion 306b of the second image 304b with the nonoccluded portion of the first image 302b. For example, the computing device extracts the occluded portion 306b from the second image 304b and replace the occluded portion 306b with the nonoccluded portion of the first image 302b. In another example, the computing device generates a composite image by overlaying the nonoccluded portion of the first image 302b on the occluded portion 306b of the second image 204b. The composite image 310b may be generated substantially in real-time. The system may determine a scale and pose adjustment factor between the real time ultrasound stream (e.g., the second image 304b) and the previously acquired CT image (e.g., the first image 302b) and transform a portion of the previously acquired CT image (e.g., the first image 302b) based on the scale and pose adjustment. The nonoccluded portion of the first image 302b may be deformed to align with the occluded portion 306b of the second image 304b. In certain examples, the nonoccluded portion may be identified within a first image 302b that was not taken at precisely the same navigational position and orientation as the second image 304b. In these examples, the computing device can either transpose the nonoccluded portion of the first image based on a comparison of navigation data between the first image 302b and the second image 304b, or interpolate between the nonoccluded portion of the first image 302b and the occluded portion 306b of the second image 304b based on the navigation data for each image. In the case of transposing the data, the nonoccluded portion is from a first image 302b that is sufficiently close in position and orientation, that the nonoccluded portion just needs to be shift into the position of the occluded portion 306b of the second image 304b. Interpolation may need to occur when the position and orientation between the first image 302b and the second image 304b is more significant (but still within the range that allows for representative adjustment of the nonoccluded portion).

At 312a, the computing device displays the composite image 310b. Despite the real-time second image having the occluded portion 306b, the user is now shown an image representation of tissue within the entire field of view as if the occluded portion 306b did not have the effect of occluding the second image 304b. If the computing device does not receive an image with an occluded portion 306b, the first image 302b is displayed unaltered by the processing described here. In an example, the computing device enables the user to switch between multiple image display modes. For example, the computing device may include a first mode that generates a composite image when an occluded portion is detected and a second mode that displays an unaltered real-time image. In some embodiments, the system may be configured to display graphical user interface elements (GUIs) indicating a delineation between a portion of the image stream which corresponds to unaltered real-time image data and which portion corresponds to composited image data that supplements the occluded portion. Exemplary such GUIs include, but are not limited to, a colored boundary line delineating the composited image portion, or, displaying the composited image portion is a different color or shade as compared to the unaltered image portion, or both.

Method 300a operates on the computing device in real-time and continuously throughout the procedure. For example, as an instrument is extended into the field-of-view, second images 304b continue to be collected with an occluded portion 306b, and method 300a operates in real-time to generate and display composite images 310b using a portion of the first image 302b extracted to replace the occluded portion 206b of the second image 304b. As another example, as the imaging device moves through a passageway, as an air bubble sits in the passageway, second images 304b continue to be collected with an occluded portion 306b, and method 300a operates in real-time to generate display composite images 310b using a portion of the first image 302b extracted to replace the occluded portion 306b of the second image 304b. First images 302b and second images 304b may be continuously received and new composite images may be displayed throughout the procedure. As another example, all of the available first images 302b from the first imaging modality are received at one time. As another example, the computing device receives the first images 302b of the target area, but additional images are available and can be received at a second time if needed. As method 300a operates, the first image 302b may be selected such that it is an image received just prior to the second image 304b, which includes the occluded portion 306b. Among other benefits, the techniques described herein enable a user to see the entire boundary of a target tissue (e.g., a SPN) even while a sampling needle or ablation device is inserted partially into the target tissue to obtain a biopsy or to perform an ablation procedure. In this way, the user is provided with valuable information within the displayed images which conventional medical imaging systems (e.g., EBUS systems) lack capabilities to provide.

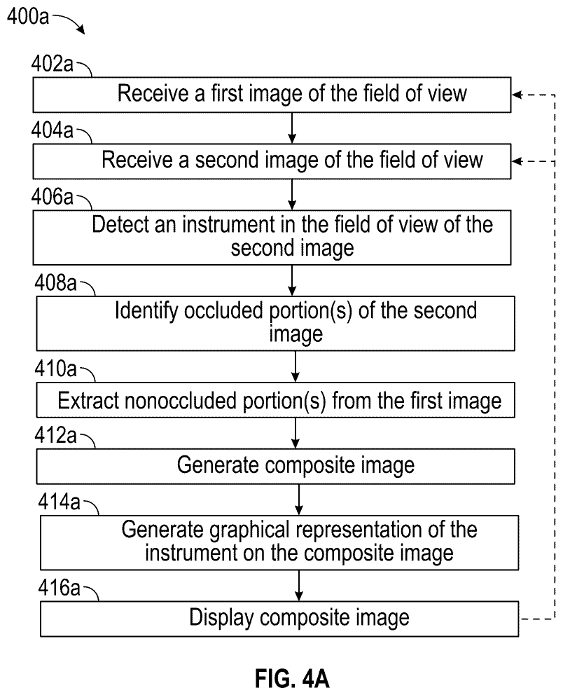

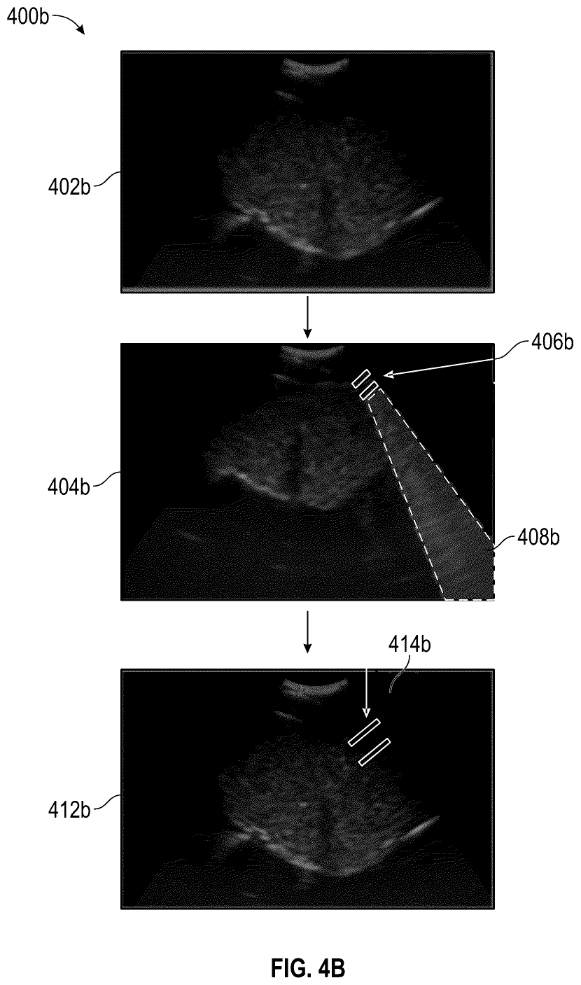

FIG. 4A illustrates an example of a method 400a of generating a composite image with a graphical indication. FIG. 4B illustrates an example set of images 400b being processed into a composite image using the method of FIG. 4A. In an example, the method 400a can be performed by a computer device (e.g., the machine of FIG. 6) communicatively coupled to the EBUS. The computer device includes processing circuitry for conducting various image processing tasks discussed in conjunction with method 400a. Further, the computer device can include an output display, such as a monitor to display the ultrasound image and various additional user interface elements, among other things.

At the beginning of method 400a, such as at the beginning of a procedure, the imaging device (e.g., the internal medical imaging system 104) is positioned to image the desired field of view. In an example, the imaging device 104 is an EBUS device with navigation capabilities such as, for example, embedded sensor coils that when placed within an electromagnetic field generated during the procedure provide signals to a navigation system that allows for precise tracking of position and orientation within the patient. The navigation system of the imaging device can utilize electromagnetic (EM) tracking techniques, that allow for tracking the imaging device within six degrees of freedom. In an example, the device is rotated between 15 degrees and 360 degrees. In an example, images are created between the images collected at different degrees using interpolation. In an example, the tracked positions include a position of the imaging device and an orientation of the imaging device.

In an example, the imaging system, or another connected system, monitors and collects respiratory information associated with the image. For example, the portion of the respiratory cycle is captured. In an example, the respiratory information is received via a respiratory gate tracking device. For example, the respiratory gate tracking device includes a plurality of markers that can move and change orientation and shape during movement of the patient caused by different stages of the respiratory cycle. In an example, the computing device uses the distance between a set of the plurality of markers at a given time to determine the stage of the respiratory cycle. In an example, the markers are visible in images. For example, the computing device uses the images of the markers to determine the distance between the markers to determine the current stage of the respiratory cycle. Accordingly, in an example, within certain parts of the anatomy, the respiratory cycle is factored into the navigation data to further enhance the orientation and position information used for imaging.

At 402a, the computing device receives a first image 402b of the field of view. The computing device receives the first image 402b captured from the imaging sensor 106 of the internal medical imaging system 104. In an example, the first image 402b is of the target area of the patient. For example, the first image 402b may include within a field of view a target tissue for which a biopsy sample and/or ablation treatment is desired such as a solitary pulmonary nodule (SPN) located just outside of and adjacent to an airway. The first image 402b may be free of any occlusions. In an example, the first image 402b includes a tracked position. In an example, the computing device receives a plurality of first images 402b. The EBUS device is a real-time imaging device, that provides a stream of images. Accordingly, in these examples, discussion of “a first image” is used generally to reference a first imaging state (e.g., a first imaging state that does not include an occlusion). Thus, when the method 400a is discussed as “receiving a first image”, this can be interpretated as a shorthand for a stream of images in a first imaging state. In certain scenarios, the computing device may operate on a single image or a stream of images with a real-time or near real-time update to the display device. For convenience, much of the method is described in terms of individual images, but the operations are performed in real-time on a stream of images.

At 404a, the computing device receives a second image 404b of the field of view. The second image 204b is received after the first image 402a. The second image 404b includes an occluded area 406b. In an example, the computing device receives the second image 404b in real time. In an example, the computing device may receive a plurality of second images 404b. In an example, computing device receives the second image 404b immediately following the first image 402a. In an example the second image 404b includes the tracked position. The second image 404b which includes the occluded area 406b may be a sequential image from the same image stream in which the first image 402b is received. For example, each of the first image 402b and second image 404b may be provided from an EBUS sampling device to an ultrasound image processor within the same image stream during a single procedure.

At 406a, the computing device detects an instrument 406b in the field of view of the second image 404b. The instrument 406b can be the instrument 120 of FIGS. 1A and 1B. If the instrument 406b is a needle, the needle can cause bright lines in the second image 404b. For example, a biopsy needle will typically produce a line of bright white pixels within an ultrasound image, as the metal needle reflects ultrasound efficiently. The bright lines are used to detect the instrument 406b. In an example, the internal medical imaging system 104 includes a sensor near the exit port 114 such that the sensor can detect when the instrument 120 is exiting the internal medical imaging system 104. In an example, the sensor is the imaging sensor 106 of the internal medical imaging system 104. For example, the computing device can use control information to detect the instrument 406b. The computing device can detect that a portion of the instrument 406b is extended into the field of view of the second image 404b. In an example, the computing device uses artificial intelligence, machine learning, or other imaging techniques to determine the instrument 406b is extended from the internal medical imaging system 104 and therefore occluding a portion of the field of view. For example, the computing device detects the instrument 406b by identifying a known instrument signature within the field of view of the second image 404b. In addition to determining the instrument 406b has been deployed or extended into the field of view, the computing device can determine a length that the instrument 406b is extended from the internal medical imaging system 104. As another example, the computing device applies a machine learning model trained to detect known image signatures generated by the instrument 406b.

At 408a, the computing device identifies one or more occluded portions 408b of the second image 404b. In an example, the computing device identifies the one or more occluded portions 408b by assessing pixels of the second image 404b. In an example, the computing device determines that the pixels in the occluded portion 408b are all black or otherwise contain data that is clearly not from imaged tissue. In another example, the computing device compares a set of pixel data from the second image 404b to a baseline value of pixel data. The baseline value of pixel can be set to indicate that the pixel is occluded. In an example, the computing device determines that a portion of the second image 404b is occluded when a subset of the set of pixel data is below the baseline value. In an example, instead of assessing individual pixels, the computing device compares pixel data in groups of pixels. The computing device can determine that an area behind the instrument 406b is the occluded portion 408b. For example, the computing device determines that beyond a certain line of data, additional information is not being collected. For example, the computing device determines that beyond the instrument 406b, additional information is not being collected. In an example, the computing device detects an immediate change in quality past an occlusion causing the occluded portion 408b (e.g., collected ultrasound data may indicate that beyond a certain depth at which a large ultrasound reflection occurs very little ultrasound data is being reflected back to the transducer). The occluded image portion may also be identified through identification of the instrument 406b within the image. For example, a biopsy needle will typically produce a line of bright white pixels within an ultrasound image, as the metal needle reflects ultrasound efficiently. In an example, the computing device examines the second image 404b in segments based on imaging rays and identify the occluded portion 408b when data is expected in the second image 404b, but no data is collected (e.g., the second image 404b is producing zeros beyond the instrument 406b). In an example, the instrument 406b (e.g., a sheath) includes an echogenic feature to enhance the detectability of the instrument 406b once imaged. For example, the echogenic feature is highly reflective. As another example, the echogenic feature is an annular ring structure. Echogenic features may damage image quality and add costs of manufacturing the instrument. In an example, when the instrument 406b does not include echogenic feature, image signatures of the sheath can be used to detect the instrument 406b in the second image 404b. In an example, the computing device detects the occluded portion 408b by detecting the instrument 406b in the field of view of the second image 404b. The computing device detects the instrument 406b using the techniques described above. In an example, the computing device detects the instrument 406b by applying an image processing algorithm trained to detect an imaging signature of an instrument. Image processing algorithms can be utilized to identify these known artifacts and can then interpolate the occluded area 408b. In certain examples, imagine processing algorithms can look for changes in contrast, such as areas with little to no contrast changes are likely to be occluded areas of an image.

At 410a, the computing device extracts the nonoccluded portion of the field of view from the first image 402b. As the imaging sensor (e.g., a transducer) may be small, the instrument 406b may cause varying sizes of occluded portions 408b, depending on how far out the instrument 406b has been deployed. However, a full two-dimensional (2D) imaging slice may be desired to aid in performing a procedure (e.g., image collection, tissue sampling). By extracting the nonoccluded portion of the first image 402b corresponding to the occluded portion 408b of the second image 404b, the occluded portion(s) 408b may be filled to show what the image would look like had the occlusion (e.g., the instrument 406b, an air bubble) not hindered imaging. The nonoccluded portion from the first image 402b substantially corresponds with the occluded portion 408b of the second image 404b. Each of the first image 402b and the second image 404b includes position and orientation data (e.g., tracked position) associated with the image. In an example, the computing device utilizes information about the field of view and the tracked position to understand a relationship between the first image 402b and the second image 404b. As another example, the computing device utilizes a plurality of first images to determine the nonoccluded portion of the first of view to extract from a first image 402b that has a substantially similar tracked position as the second image 404b. In certain examples, the nonoccluded portion may be identified within a first 402b image that was not taken at precisely the same tracked position as the second image 404b. In such examples, position and orientation data associated with the second image 404b may be used to identify precisely where in 3D space the patient's tissue is occluded and position and orientation data associated with the first image 402b may be used to identify non-occluded image data corresponding to this 3D space for use in compositing into the second image 404b.

At 412a, the computing device generates a composite image 412b. In an example, the computing device generates a composite image by replacing the occluded portion 408b of the second image 404b with the nonoccluded portion of the first image 402b. For example, the computing device generates the compose image 412b by extracting the nonoccluded portion of the first image 402b and replace the occluded portion 408b of the second image 404b. In another example, the computing device generates the composite image 412b by overlaying the nonoccluded portion of the first image 402b on the occluded portion 408b of the second image 404b. The computing device may generate the composite image 412b in substantially real-time. The computing device may determine a scale and pose adjustment factor between the real time ultrasound stream (e.g., the second image 404b) and the previously acquired ultrasound image (e.g., the first image 402b) and transform a portion of the previously acquired ultrasound image (e.g., the first image 402b) based on the scale and pose adjustment. The nonoccluded portion of the first image 402b can be deformed to align with the occluded portion 408b of the second image 404b. In certain examples, the nonoccluded portion can be identified within a first image 402b that was not taken at precisely the same navigational position and orientation as the second image 404b. In these examples, the computing device can either transpose the nonoccluded portion of the first image 402b based on a comparison of navigation data between the first image 402b and the second image 404b, or interpolate between the nonoccluded portion of the first image 402b and the occluded portion 408b of the second image 404b based on the navigation data for each image. In the case of transposing the data, the nonoccluded portion is from a first image 402b that is sufficiently close in position and orientation, that the nonoccluded portion just needs to be shift into the position of the occluded portion 408b of the second image 404b. Interpolation may need to occur when the position and orientation between the first image 402b and the second image 404b is more significant (but still within the range that allows for representative adjustment of the nonoccluded portion).

At 414a, the computing device generates a graphical representation 414b of the instrument 406b on the composite image 412b. In an example, the graphical representation 414b dynamically changes as the instrument 406b continues to be extended. For example, as the computing device receives new second images 404b, the composite image 412 may be updated with the new second image 404b, an updated nonoccluded portion from the first image 402b, and an updated graphical representation 414b. In an example, if the second image 404b has not substantially changed, and the same first image 402b is identified to be used, the computing device predicts the occluded portion 408b based on the instrument 406b and creates a composite image 412b that replaces the entire predicted occluded portion. As such, the composite image 412b may stay the same, while the graphical representation 414b of the instrument is continuously updated as the instrument 406b is further extended and/or retracted. In an example, the graphical representation 414b looks substantially similar to the instrument 406b. For example, the graphical representation 414b is shaped to look like a needle. As another example, the graphical representation 414b is shaped to look like a sheath. In another example, the graphical representation 414b indicates both the needle and the sheath. In an example, the graphical representation 414b provides an indication of how far the instrument 406b has been extended from the internal medical imaging system 104. In an example, the graphical representation 414b provides an indication that the instrument 406b is extended from the internal medical imaging system a predetermined amount. Such a graphical representation can aid in ensuring a sheath is extended a predetermined amount before extending a needle. In an example, the graphical representation 414b includes a numerical indication of an extension length of the instrument 406b. in an example, the graphical representation 414b includes a color coding corresponding to a predetermined extension length of the instrument 406b.

At 416a, the computing device displays the composite image 412b. Despite the real-time second image 404b having the occluded portion 408b, the user is now shown an image representation of tissue within the entire field of view as if the occluded portion 408b did not have the effect of occluding the second image 404b. If the computing device does not receive an image with an occluded portion 408b, the first image 402b is displayed unaltered by the processing described here. In an example, the computing device enables the user to switch between multiple image display modes. For example, the computing device may include a first mode that generates a composite image when an occluded portion is detected and a second mode that displays an unaltered real-time image. In some embodiments, the system may be configured to display graphical user interface elements (GUIs) indicating a delineation between a portion of the image stream which corresponds to unaltered real-time image data and which portion corresponds to composited image data that supplements the occluded portion. Exemplary such GUIs include, but are not limited to, a colored boundary line delineating the composited image portion, or, displaying the composited image portion is a different color or shade as compared to the unaltered image portion, or both.

Method 400a operates on the computing device in real-time and continuously throughout the procedure. For example, while the instrument 406b is extended into the field-of-view second images 404b continue to be collected with an occluded portion 408b, and method 400a operates in real-time to generate and display composite images 412b using a portion of the first image 402b extracted to replace the occluded portion 408b of the second image 404b. As another example, first images 402b will continue to be collected until a second image 404b with an occluded portion 408b is identified. First images 402b and second images 404b may be continuously received and new composite images 412b may be displayed throughout the procedure. As method 400a operates, the first image 402b may be selected such that it is an image received just prior to the second image 404b, which includes the occluded portion 406b. Among other benefits, the techniques described herein enable a user to see the entire boundary of a target tissue (e.g., a SPN) even while a sampling needle or ablation device is inserted partially into the target tissue to obtain a biopsy or to perform an ablation procedure. In this way, the user is provided with valuable information within the displayed images which conventional medical imaging systems (e.g., EBUS systems) lack capabilities to provide.

The steps or operations of the methods 200a, 300a, and 400a are illustrated in a particular order for convenience and clarity; many of the discussed operations can be performed in a different sequence or in parallel without materially impacting other operations. The methods 200a, 300a, and 400a as discussed includes operations performed by multiple different actors, devices, and/or systems. It is understood that subsets of the operations discussed in the methods 200a, 300a, and 400a can be attributable to a single actor, device, or system could be considered a separate standalone process or method.

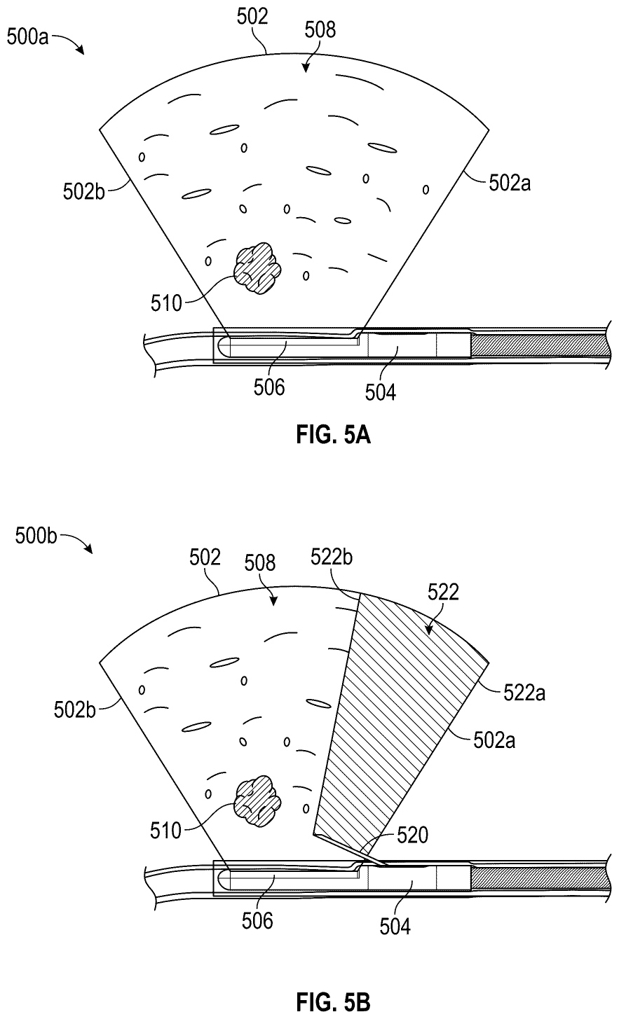

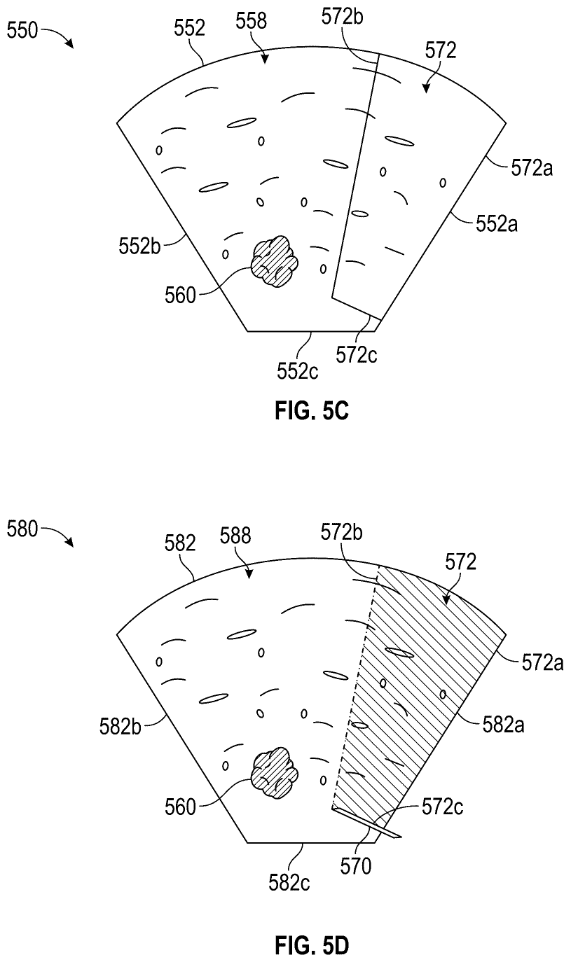

FIG. 5A a schematic diagram 500a of a nonoccluded field of view 502 from an imaging sensor 506 of an internal medical imaging system 504. In an example, the internal medical imaging system 504 is inserted into a target area of a patient. The medical imaging system 504 is comparable to the medical imaging systems discussed above.

In an example, the internal medical imaging system 504 is an endobronchial ultrasound (EBUS) tissue sampling device. In an example, the internal medical imaging system 504 is used to navigate towards and image a target nodule 510 prior to tissue sampling. In an example, the target nodule 510 is a preidentified tissue for which a biopsy sample is desired. In an example, ultrasound (US) imaging is used in the respiratory area to positively identify the target area from which to acquire a biopsy sample because the ultrasound imaging shows both tissue and an instrument (e.g., needle or sheath) in real time.

In an example, the imaging sensor 506 is positioned at a distal end of the internal medical imaging system 504. In an example, the imaging sensor 506 extends along a portion of the distal end. In an example, the imaging sensor 506 is substantially flat. In an example, the imaging sensor 506 is an ultrasound transducer. In an example, the imaging sensor 506 provides a field of view 502 to an imaging display.

The field of view 502 of the imaging sensor extends from a proximal boundary 502a to a distal boundary 502b. In the illustrated example, the entirety of the field of view 502 is a nonoccluded field of view 508.

FIG. 5B illustrates a schematic diagram 500b of a needle 520 causing an occluded portion 522 of the field of view 502 of the internal medical imaging system 504. In an example, the internal medical imaging system 504 is inserted into a target area of a patient.

In an example, the needle 520 is advanced through the internal medical imaging system 504 via a working channel. In an example, the needle 520 is extended from an exit port of the internal medical imaging system 504. In an example, the exit port includes a ramp, such that the needle 520 extends from the internal medical imaging system 504 at an angle. In an example, the needle 520 is a sampling needle for capturing biopsies of target tissues (e.g., nodule 510). For example, the target tissue be preidentified via a pre-operative image source such as a CT scan. In an example, the internal medical imaging system 504 includes a sheath to be extended from the exit port. In an example, the sheath is a flexible plastic sleeve. As an example, the sheath enshrouds the needle 520. As another example, the sheath is partially extended out of the internal medical imaging system 504 prior to the needle 520 being extended from the exit port, such that the sheath protects the exit port.

During imaging, the extension of the needle 520 out of the internal medical imaging system 504 causes an occluded portion 522 in the field of view 502, in addition to the nonoccluded portion 508. The occluded portion 522 extends from a proximal boundary 522a to a distal boundary 522b. The occluded portion 522 is caused by an interruption of the transmission of ultrasound energy to tissue beyond the needle 520 in relation to the imaging sensor 506 (e.g., US transducer). For example, ultrasonic waves transmitted from the imaging sensor 506 (e.g., an ultrasound transducer) may propagate through tissues until reaching a boundary between tissue and the needle 520 at which a substantial amount of the ultrasound energy is reflected back toward the imaging sensor 506 with an insubstantial amount continues propagating away from the imaging sensor 506 and into the needle 520. While it can be advantageous for the user to see the needle 520 within the field of view 502, the occluded portion 522 caused by the needle 520 may lack information indicating tissue characteristics beyond the needle 520. In this example, the field of view 502 and the occluded portion 522 share a common proximal boundary-proximal boundary 502a and proximal boundary 522a—while the distal boundary 522b of the occluded portion 522 will track distally as the needle 520 is extended into the field of view 502. Such a lack of information can affect the ability of a physician to direct the internal medical imaging system 504 toward a target area of the patient and/or may affect the ability of the physician to direct the needle 520 to a target nodule 510 within the target area.

In an example, the internal medical imaging system 504 of FIGS. 5A and 5B includes or is connected to a display device to display an image of the field of view 502 obtained by the imaging sensor 506. In an example, the internal medical imaging system 504 is connected to a computing device. The computing device is capable of image processing (e.g., the machine of FIG. 6).

FIG. 5C illustrates a schematic diagram 550 the field of view 552 of FIG. 5A, with a corresponding nonoccluded portion 572 identified by a computing device. As the computing device receives imaging from the internal medical imaging system, the computing device determines when a portion of an image is occluded. In an example, the computing device identifies the one or more occluded portions of the image received in real-time by assessing pixels of the field of view, identifying the needle within the field of view, identifying an area behind the needle in the field of view, determining a change in quality in a portion of the field of view of the image real-time image. Such methods are described above.

Once an occluded portion is detected, the computing device can retrieve an image received prior to the image with the occluded portion. For example, the field of view 552 of the previously acquired image can extend from a proximal boundary 552a to a distal boundary 552b and may include the target nodule 560. The computing device identifies a nonoccluded portion 572 of the previously acquired image that corresponds to an occluded portion of the real-time image. For example, the corresponding nonoccluded portion 572 aligns with the occluded portion 522 of FIG. 5B. The corresponding nonoccluded portion 572 extends from a proximal boundary 572a to a distal boundary 572b. In the illustrated example, the corresponding nonoccluded proximal boundary 572a and the field of view proximal boundary 558a are the same. The previously acquired image also includes a nonoccluded portion 558 that corresponds to a nonoccluded portion of the real-time time. As the corresponding nonoccluded portion 572 is located behind the needle, the corresponding nonoccluded portion 572 also includes a lower boundary 572c that is not aligned with a lower boundary 552c of the field of view 552, but instead follows the trajectory of a needle.

After the computing device identifies the corresponding nonoccluded portion 572 of the previously acquired image, the computing device extracts the corresponding nonoccluded portion 572 of the previously acquired image. By extracting the corresponding nonoccluded portion 572 of the previously acquired image, the occluded portion may be filled to show what the image would look like had the occlusion (e.g., the needle) not hindered imaging.

FIG. 5D illustrates a schematic diagram 580 of a composite image 582 created from the field of view of FIG. 5A and FIG. 5B. A computing device displays the composite image 582. Accordingly, the composite image 582 is the image that is displayed to the user.

As the computing device receives imaging from the internal medical imaging system, the computing device determines when a portion of a real-time image is occluded. In an example, the computing device identifies the one or more occluded portions of the image received in real-time by assessing pixels of the field of view, identifying the needle within the field of view, identifying an area behind the needle in the field of view, determining a change in quality in a portion of the field of view of the image real-time image. Such methods are described above.

The composite image 582 extends from a proximal boundary 582a to a distal boundary 582b and may include the target nodule 560. The composite image 582 includes a nonoccluded portion 588 and an occluded portion 572. The computing device uses the non-occluded portion 588 of the real-time image. Because a portion of the real-time image is occluded, the computing device uses a corresponding nonoccluded portion 572 from a previous image (e.g., corresponding nonoccluded portion 572 as shown in FIG. 5C), to create the composite image 582. The nonoccluded portion 572 extends from a proximal boundary 572a to a distal boundary 572b. In the illustrated example, the nonoccluded proximal boundary 572a and the field of view proximal boundary 582a are the same. As the nonoccluded portion 572 is located behind the needle, the nonoccluded portion 572 also includes a lower boundary 572c that is not aligned with a lower boundary 582c of the field of view 582, but instead follows the trajectory of a needle.

In an example, the computing device generates the composite image 582 by replacing the occluded portion of the real-time image with the corresponding nonoccluded portion 572 of a previously acquired image. For example, the computing device generates the compose image 582 by extracting corresponding nonoccluded portion 572 of the previously acquired image and replace the occluded portion of the real-time image. In another example, the computing device generates the composite image 582 by overlaying the corresponding nonoccluded portion 572 of the previously acquired image on the occluded portion of the real-time image. The computing device may generate the composite image 582 in substantially real-time.

In the illustrated example, the composite image 582 includes a graphical representation 570 of the needle. The computing device generates the graphical representation 570. In an example, the graphical representation 570 dynamically changes as the needle continues to be extended. For example, as the computing device receives a real-time image, the composite image 582 may be updated with the nonoccluded portion 588 of the real-time image, an updated corresponding nonoccluded portion 572 from the previously acquired image, and an updated graphical representation 570. In an example, the graphical representation 570 looks substantially similar to the needle. For example, the graphical representation 570 is shaped to look like a needle. In an example, the graphical representation 570, the composite image 582, or a graphical user interface that is displaying the composite image 582, provides an indication of how far the needle has been extended from the internal medical imaging system. In an example, the graphical representation 570, the composite image 582, or a graphical user interface that is displaying the composite image 582, provides an indication that the needle is extended from the internal medical imaging system a predetermined amount. In an example, the graphical representation 570 includes a numerical indication of an extension length of the needle. in an example, the graphical representation 570 includes a color coding corresponding to a predetermined extension length of the needle.

FIG. 6 illustrates a block diagram of an example machine 600 upon which any one or more of the techniques (processes) discussed herein may perform in accordance with some embodiments. In alternative embodiments, the machine 600 may operate as a standalone device and/or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NFC), etc.) connection to communicate and/or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600, and includes digital or analog communications signals or other intangible medium to facilitate communication of such software.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### EXAMPLES

The following Examples provide an overview of essential aspects and embodiments of the systems and methods for compositing pre-acquired image data into occluded areas of a real-time image stream. The examples are written in plain language in order to describe key features of the invention. While the examples do not delineate the full scope of the invention as defined in the claims, they are intended to highlight certain inventive concepts, components, steps and advantages in a simplified and non-limiting manner. The examples cover real-time compositing techniques to fill ultrasound image occlusions, multi-modality image compositing methods, graphical occlusion indicators, and related concepts disclosed herein. These examples are intended to supplement the technical details provided elsewhere in the specification.

Example 1 is a method for real-time replacement of occluded portions of an ultrasound imaging stream. The method can include receiving a first image of a field of view, receiving second images of the field of view that are subsequent to the first image and include occluded areas, identifying the occluded portions, extracting nonoccluded portions of the first image corresponding to the occlusions, generating composite images by replacing the occluded portions with the nonoccluded portions, and displaying the composite images in real-time.

Example 2 includes the subject matter of Example 1, with the additional feature of identifying occluded portions by detecting an instrument in the field of view.

Example 3 includes the subject matter of Example 2, with the additional feature of detecting the instrument by identifying a known instrument signature.

Example 4 includes the subject matter of Example 3, with the additional feature of detecting the instrument by applying a machine learning model trained on instrument signatures.

Example 5 includes the subject matter of any one of Examples 2-4, with the additional feature of detecting the instrument based on sensor or control information indicating part of the instrument was extended into the field of view.

Example 6 includes the subject matter of any one of Examples 2-5, with the additional feature of generating a graphical representation of the instrument on the composite images, where the representation dynamically changes to show instrument extension length.

Example 7 includes the subject matter of Example 6, with the additional feature of displaying an indication that the instrument has extended a predetermined amount.

Example 8 includes the subject matter of Example 7, with the feature that the indication is a numerical value of the extension length.

Example 9 includes the subject matter of Example 7, with the feature that the indication uses color coding based on the extension length.

Example 10 includes the subject matter of any one of Examples 1-9, with the additional feature of identifying occluded portions by detecting air bubbles using image processing algorithms.

Example 11 includes the subject matter of any one of Examples 1-10, with the additional feature of identifying occluded portions by comparing pixel data to baseline levels and determining occluded areas based on pixels below the baseline levels.

Example 12 includes the subject matter of any one of Examples 1-11, with the additional feature of tracking position and orientation of the first and second images.

Example 13 includes the subject matter of Example 12, with the additional feature of deforming the nonoccluded portion of the first image when generating the composite images.

Example 14 includes the subject matter of Example 13, with the additional feature of deforming the nonoccluded portion by interpolating to account for differences in position and orientation between the first and second images.

Example 15 is an internal medical imaging system including an imaging sensor, display device, and computing device. The computing device can receive first and second images, identify occluded portions of the second images, extract nonoccluded portions of the first images, generate composite images, and display the composite images in real-time.

Example 16 includes the subject matter of Example 15, with the additional feature of the computing device detecting an instrument in the field of view to identify occluded portions.

Example 17 includes the subject matter of Example 16, with the additional feature of detecting the instrument by identifying known instrument signatures.

Example 18 includes the subject matter of Example 17, with the additional feature of detecting the instrument by applying an instrument signature detection model.

Example 19 includes the subject matter of any one of Examples 16-18, with the additional feature of detecting the instrument based on sensor or control data indicating part of the instrument extended into the field of view.

Example 20 includes the subject matter of any one of Examples 16-19, with the additional feature of displaying a graphical representation of the instrument on the composite images.

Example 21 includes the subject matter of Example 20, with the additional feature of displaying an indication that the instrument has extended past a predetermined amount.

Example 22 includes the subject matter of Example 21, with the feature that the indication is a numerical value of the extension length.

Example 23 includes the subject matter of Example 21, with the feature that the indication uses color coding based on the extension length.

Example 24 includes the subject matter of any one of Examples 15-23, with the additional feature of identifying occluded portions by detecting air bubbles using image processing algorithms.

Example 25 includes the subject matter of any one of Examples 15-24, with the additional feature of identifying occluded portions by comparing pixel data to baseline levels and determining occluded areas based on pixels below the baseline levels.

Example 26 includes the subject matter of any one of Examples 15-25, with the additional feature of the computing device tracking position and orientation of the first and second images.

Example 27 includes the subject matter of Example 26, with the additional feature of the computing device deforming the nonoccluded portion of the first image when generating the composite images.

Example 28 includes the subject matter of Example 27, with the additional feature of deforming the nonoccluded portion by interpolating to account for differences in position and orientation between the first and second images.

Example 29 is a method for real-time replacement of ultrasound image occlusions using multi-modality images. The method can include receiving a previously acquired CT image, receiving a live ultrasound image with occlusions, extracting a nonoccluded CT portion, and updating the live ultrasound image by overlaying the nonoccluded CT portion to generate an augmented live image.

Example 30 includes the subject matter of Example 29, with the additional feature of deforming the nonoccluded CT portion to match the anatomical state of the live ultrasound image.

Example 31 includes the subject matter of Example 30, with the additional feature of deforming the CT portion by identifying anatomical landmarks in the CT and ultrasound images.

Example 32 includes the subject matter of any one of Examples 30-31, with the additional feature of deforming the CT portion based on tracking respiratory state.

Example 33 includes the subject matter of Example 32, with the additional feature of accounting for differences in respiratory state between the CT image and live ultrasound image when deforming the CT portion.

Example 34 includes the subject matter of Example 32, with the additional feature of selecting a CT image with a respiratory state matching that of the live ultrasound image.

Example 35 includes the subject matter of any one of Examples 30-34, with the additional feature of deforming the CT portion by interpolating to account for positional and orientational differences between the CT and ultrasound images.

Example 36 includes the subject matter of any one of Examples 29-35, with the additional feature that the previously acquired image is a CT image.

Example 37 includes the subject matter of any one of Examples 29-36, with the additional feature that overlaying the nonoccluded CT portion generates an augmented live ultrasound image.

1. An internal medical imaging system comprising:
an imaging sensor configured to obtain medical images within an internal portion of a patient;
a display device configured to display image data and related graphical information from the imaging sensor; and
a computing device including a processor and a memory device, the memory device including instructions that, when executed by the processor, cause the computing device to perform operations including:
receive, from the imaging sensor, at least one first image of a field of view of a patient;
receive, from the imaging sensor, a plurality of second images of the field of view, the plurality of second images received subsequent to the at least one first image;
identify an occluded portions of the plurality of second images within the field of view;
extract at least one nonoccluded portion of the at least one first image corresponding to the occluded portions;
generate a plurality of composite images by replacing the occluded portion of the second image with the at least one nonoccluded portion from the at least one first image; and
send, to the display device, the plurality of composite images in substantially real-time as the receiving the plurality of second images.

2. The internal medical imaging system of claim 1, further comprising an instrument, wherein the instructions further cause the computing device to perform operations including identify the occluded portions of the plurality of second images by detecting the instrument in the field of view.

3. The internal medical imaging system of claim 2, wherein the detecting the instrument in the field of view includes identifying a known instrument signature within the field of view.

4. The internal medical imaging system of claim 3, wherein the detecting the instrument in the field of view includes applying a machine learning model trained to detect known image signatures generated by the instrument.

5. The internal medical imaging system of claim 2, wherein the instrument further comprises a sensor or controller and wherein the instructions further cause the computing device to perform operations including detect the instrument in the field of view by receiving sensor or control information from the instrument indicating that a portion of the instrument has been extended into the field of view.

6. The internal medical imaging system of claim 2, wherein the instructions further cause the computing device to perform operations including generate the plurality of composite images by generating a graphic indicating the instrument causing the occluded portions of the plurality of second images.

7. The internal medical imaging system of claim 6, wherein the instructions further cause the computing device to perform operations including display the plurality of composite images by displaying an indication that the instrument is extended a predetermined amount.

8. The internal medical imaging system of claim 7, wherein the indication is a numerical indication of an extension length of the instrument.

9. The internal medical imaging system of claim 7, wherein the indication is a graphical indication including a color coding corresponding to a predetermined extension length of the instrument.

10. The internal medical imaging system of claim 1, wherein the instructions further cause the computing device to perform operations including identify the occluded portions of the plurality of second images by detecting an air bubble in the field of view.

11. The internal medical imaging system of claim 10, wherein the instructions further cause the computing device to perform operations including detect the air bubble in the field of view by applying an image processing algorithm trained to detect an ultrasound signature of an air bubble.

12. The internal medical imaging system of claim 1, wherein the instructions further cause the computing device to perform operations including identify the occluded portions of the plurality of second images by comparing a set of pixel data of the plurality of second images to a baseline value and determining the occluded portion when a subset of the set of pixel data is below the baseline value.

13. The internal medical imaging system of claim 1, wherein the instructions further cause the computing device to perform operations including track position and orientation of at least one first image and the plurality of second images.

14. The internal medical imaging system of claim 13, wherein the instructions further cause the computing device to perform operations including generate the plurality of composite images by deforming the nonoccluded portion of the at least one first image.

15. The internal medical imaging system of claim 14, wherein the instructions further cause the computing device to perform operations including deform the nonoccluded portion of the at least one first image by interpolating to account for differences in position and orientation of the plurality of second images and the at least one first image.

16. A method for real-time replacement of occluded portions of an ultrasound imaging stream, the method comprising:
receiving, via the ultrasound imaging stream, at least one first image of a field of view of a patient;
receiving, via the ultrasound imaging stream, a plurality of second images of the field of view, the plurality of second images received subsequent to the at least one first image;
identifying occluded portions of the plurality of second images within the field of view;
extracting at least one nonoccluded portion of the at least one first image corresponding to the occluded portions;
generating a plurality of composite images by replacing the occluded portions of the plurality of second images with the at least one nonoccluded portion from the at least one first image; and
displaying the plurality of composite images in substantially real-time as the receiving the plurality of second images.

17. The method of claim 16, wherein identifying occluded portions of the plurality of second images includes detecting an instrument in the field of view.

18. The method of claim 17, wherein detecting the instrument in the field of view comprises identifying a known instrument signature within the field of view.

19. The method of claim 18, wherein detecting the instrument in the field of view comprises applying a machine learning model trained to detect known image signatures generated by the instrument.

20. The method of claim 17, wherein detecting the instrument in the field of view comprises receiving sensor or control information from the instrument indicating that a portion of the instrument has been extended into the field of view.

21. The method of claim 17, wherein generating the plurality of composite images comprises generating a graphical representation of the instrument causing the occluded portions, wherein the graphical representation dynamically changes to represent an extension length of the instrument within the field of view.
